(19) United States
(12) Patent Application Publication
(10) Pub. No.: US 2012/0059054 A1
(43) Pub. Date: Mar. 8, 2012

(54) Use Of Copolymers Based On Amino-Containing Polymers As Matrix Binders In Preparing Active Compound-Containing Granules And Administration Forms

(75) Inventors: Karl Kolter, Limburgerhof (DE); Maximilian Angel, Schifferstadt (DE)

(73) Assignee: BASF SE, Ludwigshafen (DE)

(21) Appl. No.: 13/225,969

(22) Filed: Sep. 6, 2011

Related U.S. Application Data
(60) Provisional application No. 61/380,336, filed on Sep. 7, 2010.

(51) Int. Cl. A61K 31/375; A61P 3/02; A61P 25/04; A61K 31/167

(52) U.S. Cl. 514/474; 514/630

(57) ABSTRACT

Described are matrix binders comprising copolymers based on amino-containing polymers, as well as their use in preparing active compound-containing granules and administration forms

The copolymers having basic amino groups can be obtained by radical polymerization of:
a) N,N-diethylaminoethyl methacrylate, and
b) at least one radically polymerizable compound, selected from esters of α,β-ethylenically unsaturated mono- and dicarboxylic acids with C₁-C₈ alkanols.

### CROSS-REFERENCE TO RELATED APPLICATIONS

This application claims priority under 35 U.S.C. §119(e) to U.S. Provisional Application No. 61/380,336, filed Sep. 7, 2010, the disclosures of which are incorporated herein by reference in their entirety.

### TECHNICAL FIELD

The present invention relates to matrix binders based on a cationic polymer which is obtained by means of radical emulsion polymerization of a monomer mixture comprising N,N-diethylaminoethyl methacrylate, for producing active compound-containing granules and administration forms comprising such granules.

### BACKGROUND

Binders in pharmaceutical technology are substances which cause powder particles to adhere to one another. In connection with binders which develop the three-dimensional structure of shaped bodies such as granules or tablets, the term “matrix binders” is also used.

DE-B 1090381 describes a method of coating dosage forms with coating materials that are soluble in the stomach. These comprise an acrylate copolymer of 20-80% of at least one amino ester of (meth)acrylic acid such as for example the esters of acrylic acid and (meth)acrylic acid with N,N-dimethylaminoethanol, N,N-diethylaminoethanol, N,N-dimethylaminopropanol and N-(hydroxyethyl)morpholine.

DE-B 1219175 describes coating materials based on copolymers containing N,N-dialkylaminoalkyl(meth)acrylamides incorporated by polymerization. Copolymers based on N,N-dialkylaminoalkyl(meth)acrylates are, according to the teaching of this document, regarded as disadvantageous, as the ester group, compared with the amide group, is saponified earlier in the basic environment.

DE-A 2135073 likewise describes coating materials for dosage forms that comprise an aqueous polymer dispersion, with the polymer consisting to 10-55 wt. % of monomers with a carboxyl group and/or a monoalkyl- or dialkylaminoalkyl ester group. Diethylaminoethyl methacrylate (DEAEMA) is mentioned as a suitable monomer, in addition to many others. Actual emulsion polymerizates based on DEAEMA are not disclosed.

DE-B 2512238 teaches, for the preparation of binders for pharmaceutical coatings with low residual monomer content, the use of a powder obtained by spray-drying of a polymer dispersion for the production of coating solutions for these dosage forms. Regarding the dispersions used for spray-drying, reference is made to DE 1090381, DE 1219175 and DE 2135073.

DE-A 2838278 describes coatings for oral dosage forms for ruminants from a) at least one film-forming polymer with at least one basic amino group, for example a copolymer of 40% N,N-diethylaminoethyl methacrylate, though without stating a method of production thereof.

GB 1324087 describes coating polymers for oral dosage forms for ruminants, which comprise a) at least one N,N-dialkylaminoalkyl(meth)acrylate and b) at least one ethylenically unsaturated compound, which is selected from vinylic aromatics and derivatives thereof, vinyl esters, esters of (meth)acrylic acid and acrylonitrile, incorporated by polymerization. N,N-dimethylaminoethyl methacrylate (DMAEMA) and tert-butylaminoethyl methacrylate (TBAEMA) are disclosed as suitable monomers a). In particular, methyl methacrylate is regarded as unsuitable as comonomer b), as it tends to form coatings which are too brittle. Bulk, suspension, solution and emulsion polymerization are stated as suitable methods of polymerization. The copolymers in the examples were produced by solution polymerization.

DE 3426587 A1 describes a method of coating dosage forms by applying a film of a liquid, film-forming coating material, which contains a dissolved polymerizate with tertiary ammonium salt side groups. For production of these polymer solutions, among other means, copolymers based on N,N-dialkylaminoalkyl(meth)acrylates can be transformed with aqueous inorganic or organic acids to aqueous solutions of ammonium salts.

DE 3049179 A1 is an additional application to DE 2512238 and relates to the use of a powder, obtained by spray-drying according to the teaching of the latter document, in the form of an aqueous suspension, which additionally contains a plasticizer, for the production of coatings by thermal gelation.

EP 0058765 A2 describes coating materials for dosage forms soluble or swellable in gastric juice, and comprise, as binder, an emulsion polymerizate based on N,N-dialkylaminoalkyl(meth)acrylates, with a branched alkylene or aralkylene group, with at least three carbon atoms arranged in a straight chain, being located between the amino group and the (meth)acrylate group.

WO 2005/055986 and WO 2005/056619 describe polymers with pH-dependent swelling/dissolution behavior and use thereof in dosage forms.

WO 00/05307 relates to the preparation of coating materials and binders for dosage forms, which comprise (meth)acrylate copolymers, having monomer residues with tertiary amino groups, and simple dry or aqueous further processing is said to be possible. For this, this document teaches a method in which (a) a copolymer of C₁-C₄ esters of (meth)acrylic acid and (meth)acrylate monomers, which have tertiary ammonium groups, (b) a plasticizer and (c) an emulsifier with an HLB value of at least 14, are mixed together and the coating material or binder is produced therefrom by melting, pouring, spreading or spraying, copolymer (a) being applied in the form of powder with an average particle size of 1-40 μm. The resultant processability is attributed to the provision of copolymer (a) in powder form with extremely small grain size.

WO 02/067906 relates to coatings and binders with improved permeability to water vapor relative to those described in WO 00/05307. The coatings and binders are produced with a mixture containing (a) a copolymer of C₁-C₄ esters of (meth)acrylic acid and other (meth)acrylate monomers with functional tertiary ammonium groups in powder form with an average particle size of 1-40 μm, (b) an emulsifier with an HLB value of at least 14 and (c) a C₁₂-C₁₈ monocarboxylic acid or a C₁₂-C₁₈ hydroxyl compound.

WO 2004/019918 describes coatings and binders that correspond, with respect to their composition, to those described in WO 00/05307 and WO 02/067906.

According to U.S. Pat. No. 6,696,085 B2, a methacrylic acid copolymer of type C is used as a disintegrant. The methacrylic acid copolymer of type C is an enteric polymer, which is insoluble at acid pH, but is water-soluble at pH of about 7, as in the oral cavity. In addition to a low breaking strength (<20N), the tablets have high friability (>7%) and have a high proportion of a coarse-grained disintegrant, in the region of 15 wt. %. Consequently, they have low mechanical strength and produce an unpleasant, sandy sensation in the mouth, owing to the high proportion of coarse-grained disintegrant.

The matrix components based on sugar alcohols, disintegrants and insoluble polymers are generally known for pharmaceutical applications from WO 2007/071581.

The production of the aqueous polymer dispersions of cationic polymers based on N,N-diethylaminoethyl methacrylate, as used according to the invention and their use for the coating of pharmaceuticals, is known from WO 2009/016258. It is also indicated, generally, that the polymers are suitable for coating agents and binders, although the only use specifically described is as film-formers in coating agents. There is no mention of any use as matrix binder for producing granules.

### SUMMARY

One or more embodiments of the present invention relate to matrix binders based on a cationic polymer which is obtained by means of radical emulsion polymerization of a monomer mixture comprising N,N-diethylaminoethyl methacrylate, for producing active compound-containing granules and administration forms comprising such granules.

### DETAILED DESCRIPTION

In one or more embodiments, provided are improved matrix binders which result in a strong granulation effect, a reduced fine fraction in the granules, and granules whose abrasion sensitivity is low. Administration forms produced from these granules by tabletting ought, even with a low compression pressure, to have a high strength and to disintegrate rapidly.

Accordingly, the use of copolymers as matrix binders in preparing active compound-containing granules was found, the copolymers containing, as component A, a polymer obtained by radical polymerization from:
a) N,N-diethylaminoethyl methacrylate,
b) at least one radically polymerizable compound, selected from esters of α,β-ethylenically unsaturated mono- and dicarboxylic acids with C₁-C₈ alkanols.

The active compound-containing granules are, more particularly, pharmaceutical granules.

The matrix binders, also referred to below as “binders”, may optionally further comprise:
ii) one or more antioxidants as component B,
iii) one or more plasticizers as component C, and
iv) physiologically acceptable acids as components D.

The matrix binders are used preferably as wet binders. With particular preference the wet-binder preparations are used in the form of aqueous polymer dispersions.

The binder preparations can comprise, relative to the total weight of the preparation,
i) 1-45 wt. % of component A,
ii) 0-10 wt. % of component B,
iii) 0-15 wt. % of component C,
iv) 0-35 wt. % of components D.

Preferred binder preparations comprise, relative to the total weight of the preparation,
i) 2-40 wt. % of component A,
ii) 0-10 wt. % of component B,
iii) 0-12.5 wt. % of component C,
iv) 0-30 wt. % of components D.

Especially preferred binder preparations comprise, relative to the total weight of the preparation,
i) 5-30 wt. % of component A,
ii) 0-5 wt. % of component B,
iii) 0-8 wt. % of component C,
iv) 0.1-20 wt. % of components D.

Particularly preferred, furthermore, are binder preparations comprising, relative to the total weight of the preparation,
i) 5-30 wt. % of component A,
ii) 0-5 wt. % of component B,
iii) 0.1-8 wt. % of component C,
iv) 0-20 wt. % of components D.

### Component A

### Monomer a)

N,N-Diethylaminoethyl methacrylate is used according to the invention as monomer a).

For production of the aqueous polymer dispersions Pd) according to the invention, component a) is preferably used in an amount of 25-65 wt. %, especially preferably 30-60 wt. %, in particular 38-48 wt. %, and especially 43-47 wt. %, relative to the total weight of the monomers used for polymerization.

### Monomer b)

Component b) is selected from esters of alpha, beta-ethylenically unsaturated mono- and dicarboxylic acids with C₁-C₈ alkanols.

Suitable compounds b) are methyl(meth)acrylate, methyl ethacrylate, ethyl(meth)acrylate, ethyl ethacrylate, n-propyl(meth)acrylate, isopropyl(meth)acrylate, n-butyl(meth)acrylate, sec-butyl(meth)acrylate, tert-butyl(meth)acrylate, tert-butyl ethacrylate, n-hexyl(meth)acrylate, n-heptyl(meth)acrylate, n-octyl(meth)acrylate, 1,1,3,3-tetramethylbutyl(meth)acrylate and ethylhexyl(meth)acrylate.

Preferably, methyl methacrylate or a monomer mixture containing methyl methacrylate is used as component b).

For production of the aqueous polymer dispersions according to certain embodiments of the invention, component b) is preferably used in an amount of 35-75 wt. %, especially preferably 40-70 wt. %, in particular 52-62 wt. %, and especially 53-57 wt. %, relative to the total weight of the monomers used for polymerization.

The monomer mixtures M) used for production of the polymer dispersions can additionally comprise at least one other monomer c). The additional monomers c) are preferably selected from esters of alpha, beta-ethylenically unsaturated mono- and dicarboxylic acids with C₉-C₃₀ alkanols and C₂-C₃₀ alkane diols, amides of alpha, beta-ethylenically unsaturated mono- and dicarboxylic acids with C₂-C₃₀ amino alcohols, which have a primary or secondary amino group, primary amides of alpha, beta-ethylenically unsaturated monocarboxylic acids and N-alkyl and N,N-dialkyl derivatives thereof, N-vinyllactams, open-chain N-vinylamide compounds, esters of vinyl alcohol and allyl alcohol with C₁-C₃₀ monocarboxylic acids, vinyl ethers, vinylic aromatics, vinyl halides, vinylidene halides, C₂-C₈ monoolefins, unsaturated nitriles, nonaromatic hydrocarbons with at least two conjugated double bonds and mixtures thereof.

Suitable additional monomers c) are esters of alpha, beta-ethylenically unsaturated mono- and dicarboxylic acids with C₉-C₃₀ alkanols, such as n-nonyl(meth)acrylate, n-decyl(meth)acrylate, n-undecyl(meth)acrylate, tridecyl(meth)acrylate, myristyl(meth)acrylate, pentadecyl(meth)acrylate, palmityl(meth)acrylate, heptadecyl(meth)acrylate, nonadecyl(meth)acrylate, arachinyl(meth)acrylate, behenyl(meth)acrylate, lignoceryl(meth)acrylate, cerotinyl(meth)acrylate, melissinyl(meth)acrylate, palm itoleinyl(meth)acrylate, oleyl(meth)acrylate, linolyl(meth)acrylate, linolenyl(meth)acrylate, stearyl(meth)acrylate, lauryl(meth)acrylate and mixtures thereof.

Suitable additional monomers c) are moreover esters of alpha, beta-ethylenically unsaturated mono- and dicarboxylic acids with C₂-C₃₀ alkane diols, such as 2-hydroxyethylacrylate, 2-hydroxyethyl methacrylate, 2-hydroxyethyl ethacrylate, 2-hydroxypropylacrylate, 2-hydroxypropylmethacrylate, 3-hydroxypropylacrylate, 3-hydroxypropylmethacrylate, 3-hydroxybutylacrylate, 3-hydroxybutylmethacrylate, 4-hydroxybutylacrylate, 4-hydroxybutylmethacrylate, 6-hydroxyhexylacrylate, 6-hydroxyhexylmethacrylate, 3-hydroxy-2-ethylhexylacrylate, 3-hydroxy-2-ethylhexylmethacrylate, etc.

Suitable additional monomers c) are moreover primary amides of alpha, beta-ethylenically unsaturated monocarboxylic acids and N-alkyl and N,N-dialkyl derivatives thereof, such as acrylic acid amide, methacrylic acid amide, N-methyl(meth)acrylamide, N-ethyl(meth)acrylamide, N-propyl(meth)acrylamide, N-(n-butyl)(meth)acrylamide, N-(tert-butyl)(meth)acrylamide, N-(n-octyl)(meth)acrylamide, N-(1,1,3,3 tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N pentadecyl(meth)acrylamide, N-palmityl(meth)acrylamide, N-heptadecyl(meth)acrylamide, N-nonadecyl(meth)acrylamide, N-arachinyl(meth)acrylamide, N-behenyl(meth)acrylamide, N-lignoceryl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide, N-lauryl(meth)acrylamide, N,N-dimethyl(meth)acrylamide, N,N-diethyl(meth)acrylamide, and morpholinyl(meth)acrylamide.

Other suitable additional monomers c) are N-vinyllactams and derivatives thereof, which can have e.g. one or more C₁-C₆ alkyl substituents, such as methyl, ethyl, n-propyl, isopropyl, n-butyl, sec-butyl, tert-butyl etc. These include e.g. N-vinyl pyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc. Preferably, N-vinyl pyrrolidone and N-vinylcaprolactam are used.

Open-chain N-vinylamide compounds suitable as monomers c) are, for example, N-vinylformamide, N-vinyl-N-methylformamide, N-vinylacetamide, N-vinyl-N-methylacetamide, N-vinyl-N-ethylacetamide, N-vinylpropionamide, N-vinyl-N-methylpropionamide and N-vinylbutyramide.

Suitable additional monomers c) are moreover vinyl acetate, vinyl propionate, vinyl butyrate and mixtures thereof.

Suitable additional monomers c) are moreover ethylene, propylene, isobutylene, butadiene, styrene, alpha-methylstyrene, acrylonitrile, methacrylonitrile, vinyl chloride, vinylidene chloride, vinyl fluoride, vinylidene fluoride and mixtures thereof.

The aforementioned additional monomers c) can be used individually or in the form of any mixtures.

For production of the aqueous polymer dispersions according to the invention, component c) is preferably used in an amount of 0-80 wt. %, relative to the total weight of the monomers used for polymerization. A special embodiment relates to polymer dispersions Pd), which do not comprise any additional monomer c) incorporated by polymerization. If present, component c) is preferably used in an amount of 0.1-70 wt. %, especially preferably 1-60 wt. %, in particular 5-50 wt. %, relative to the total weight of the monomers used for polymerization.

Preferably no monomer c) is used.

### Monomer d)

The monomer mixtures M) used for production of the polymer dispersions can comprise, in addition to compound a), at least one other compound d) different from the latter with a radically polymerizable alpha, beta-ethylenically unsaturated double bond and at least one cationogenic and/or cationic group per molecule, incorporated by polymerization.

Preferably, the cationogenic or cationic groups of component d) are nitrogen-containing groups, such as primary, secondary and tertiary amino groups and quaternary ammonium groups. Preferably, the nitrogen-containing groups are tertiary amino groups or quaternary ammonium groups. Charged cationic groups can be produced from the amine nitrogens either by protonation, e.g. with monovalent or polyvalent carboxylic acids, such as lactic acid or tartaric acid, or mineral acids, such as phosphoric acid, sulfuric acid and hydrochloric acid, or by quaternization, e.g. with alkylating agents, such as C₁-C₄ alkyl halides or sulfates. Examples of said alkylating agents are ethyl chloride, ethyl bromide, methyl chloride, methyl bromide, dimethyl sulfate and diethyl sulfate.

Suitable compounds d) are e.g. the esters of alpha, beta-ethylenically unsaturated mono- and dicarboxylic acids with amino alcohols that are different from DEAEMA. Preferred amino alcohols are C₂-C₁₂ amino alcohols, which are C₁-C₈-mono- or dialkylated on the amine nitrogen. For example, acrylic acid, methacrylic acid, fumaric acid, maleic acid, itaconic acid, crotonic acid, maleic anhydride, monobutylmaleate and mixtures thereof are suitable as the acid component of these esters. Preferably, acrylic acid, methacrylic acid and mixtures thereof are used as the acid component of these esters.

Suitable additional compounds d) are N,N-dimethylaminomethyl(meth)acrylate, N,N-dimethylaminoethyl(meth)acrylate, N,N-diethylaminoethylacrylate, N,N-dimethylaminopropyl(meth)acrylate, N,N-diethylaminopropyl(meth)acrylate and N,N-dimethylaminocyclohexyl(meth)acrylate.

Suitable monomers d) are furthermore the amides of the aforementioned alpha, beta-ethylenically unsaturated mono- and dicarboxylic acids with diamines, which have at least one primary or secondary amino group. Diamines that have a tertiary and a primary or secondary amino group are preferred.

These include N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)-butyl]methacrylamide, N-[2-(diethylamino)ethyl]acrylamide, N-[4-(dimethylamino)cyclohexyl]acrylamide, N-[4-(dimethylamino)cyclohexyl]methacrylamide etc.

Suitable monomers d) are furthermore N,N-diallyl amines and N,N-diallyl-N-alkyl amines and their salts of acid addition and quaternization products. Alkyl then preferably stands for C₁-C₂₄ alkyl. N,N-diallyl-N-methyl amine and N,N-diallyl-N,N-dimethylammonium compounds, e.g. the chlorides and bromides, are preferred.

Suitable monomers d) are furthermore vinyl- and allyl-substituted nitrogen heterocycles, such as N-vinylimidazole, N-vinyl-2-methylimidazole, vinyl- and allyl-substituted heteroaromatic compounds, such as 2- and 4-vinylpyridine, 2- and 4-allylpyridine, and salts thereof.

For production of the aqueous polymer dispersions Pd) according to the invention, the monomer d), if present, is preferably used in an amount such that the sum of the amounts of monomers a) and of monomers d) is in the range 25-65 wt. %, especially preferably 30-60 wt. %, relative to the total weight of the monomers used for polymerization.

For production of the aqueous polymer dispersions Pd) according to the invention, component d) is preferably used in an amount of 0-50 wt. %, relative to the total weight of the monomers used for polymerization.

As already stated, it was found, surprisingly, that the polymer dispersions Pd) according to the certain embodiments invention and used according to one or more embodiments of the invention, based on DEAEMA (component a)), have a particularly good profile of properties. This profile of properties can as a rule be achieved without using additional monomers with cationogenic/cationic groups. A special embodiment therefore relates to polymer dispersions Pd) that do not comprise any additional monomer d) incorporated by polymerization.

If present, component d) is preferably used in an amount of 0.1-40 wt. %, especially preferably 1-30 wt. %, in particular 2-25 wt. %, relative to the total weight of the monomers used for polymerization.

In an especially preferred embodiment of the method according to the invention, a monomer mixture M) is used that consists of
43-47 wt. %, relative to the total weight of the monomers used for polymerization, of N,N-diethylaminoethyl methacrylate a), and
53-57 wt. %, relative to the total weight of the monomers used for polymerization, of at least one compound b), in particular methyl methacrylate.

For production of the polymerizates by radical emulsion polymerization, reference is hereby expressly made to the disclosure of WO 2009/016258, in which the production and preferred embodiments and actual methods of production are described in detail.

The polymers contained in the dispersions according to certain embodiments of the invention preferably have an average molecular weight M_{w}, determined by gel permeation chromatography, in the range 30000-500000, especially preferably 60000-140000, in particular 80000-120000 g/mol.

The polymers contained in the dispersions Pd) preferably have a K value (determined according to Fikentscher on a 1% solution in N-methylpyrrolidone (NMP)) in the range 40-60.

The glass transition temperature T_{g} (determined by DSC) is preferably in the range 40-70° C., especially preferably 52-62° C.

In one or more embodiments, the polymers contained in the dispersions used are essentially random copolymers.

The average particle diameter of the polymer particles contained in the polymer dispersion (determined by means of an analytical ultracentrifuge) is preferably in the range from 70 to 200 nm, especially preferably from 80 to 150 nm, in particular from 90 to 120 nm. The particle size distribution is preferably substantially unimodal.

The LT value of the dispersions, determined on a 0.01% dispersion in water (2.5 cm cuvette, white light) is preferably at least 70%, especially preferably at least 80%. Determination of the light transmission is described e.g. in Dieter Distler, Aqueous Polymer Dispersions, Wiley-VCH (1999), p. 40.

The solids content of the dispersions after production is preferably 10-50 wt. %, especially preferably 20-40 wt. %. In the case of purification of the dispersion by ultrafiltration, the dispersions preferably have solid contents that are within this range before and after ultrafiltration. It is, of course, also possible for a diluted polymer dispersion to be submitted to concentration by ultrafiltration.

The dispersions used as matrix binders for granules can have, for example, even at a solids content of 30 wt. %, extremely low viscosities preferably of less than 50 mPas, especially preferably less than 25 mPas and in particular less than 10 mPas (values determined with a Brookfield viscosimeter at 20° C. and 100 s⁻¹). Such low viscosities are particularly important for many applications.

The charge of the polymers contained in the depends on the pH of the dispersion. The isoelectric point is preferably in a pH range from about 7.5 to 8.5. The prepared dispersion preferably has a pH in the range 8-10, especially preferably 8.5-9.5 (at a solids content of 30 wt. %). It is advantageous for the pH of the prepared dispersion to be selected higher (more alkaline) than its isoelectric point, unless dissolution or swelling of the polymer particles contained in the dispersion is desired. Therefore the dispersions are preferably basic dispersions.

The polymer dispersions are characterized by their pH-dependent solubility. The pH range in which the dispersion dissolves on acidification can be adjusted e.g. by the amount of N,N-diethylaminoethyl methacrylate (monomer a) incorporated by polymerization, and optionally the use of additional monomers with cationogenic/cationic groups (monomer d). Preferably the polymers contained in the polymer dispersions Pd) according to the invention dissolve at a pH of max. 6.8, especially preferably at a pH of max. 6.0.

According to a preferred embodiment, polymer dispersions are used that comprise a polymer that contains
43-47 wt. %, relative to the total weight of the monomers used for polymerization, of N,N-diethylaminoethyl methacrylate a), and
53-57 wt. %, relative to the total weight of the monomers used for polymerization, of at least one compound b)
   as the only monomers, incorporated by polymerization, the sum of a) and b) adding to 100 wt. %.

### Component B

The coating materials according to certain embodiments of the invention may comprise, in addition to the polymer, one or more antioxidants or a combination of antioxidants (indicated by “/”).

The following agents, the combinations listed or other combinations are suitable as antioxidants for improving the release stability:

N-acetylcysteine, allantoin, arginine, arginine/butyl hydroxytoluene, arginine/N-acetylcysteine, ascorbyl palmitate, aspartic acid, biotin, butyl hydroxyanisole, butyl hydroxytoluene, butyl hydroxytoluene/calcium carbonate, butyl hydroxytoluene/Na-EDTA, butyl hydroxytoluene/N-acetylcysteine, calcium-bis[monoethyl(3,5-di-tert-butyl-4-hydroxy-benzyl)phosphonate], catechol, citric acid, cysteamine, ethylhexylthioglycolate, gallic acid, hypophosphorous acid, caffeic acid, potassium iodide, creatine, creatinine, copper(I) chloride, copper(II) chloride, lysine, MEHQ, methionine, Na-EDTA, sodium carbonate, sodium dihydrogen phosphate, disodium hydrogen phosphate, sodium propionate, nordihydroguaiaretic acid, orotic acid, penicillamine, phosphoric acid, propyl gallate, resveratrol, riboflavin, spermidine, thioglycolic acid, tocopherol, tocopherol acetate, trometamol, tyrosine and tartaric acid.

The following agents, the combinations listed or other combinations are suitable for improving resistance to yellowing:

oleic acid, simethicone, butyl hydroxytoluene, sodium hydrogen sulfite, tocopherol, sodium dihydrogen citrate, sodium hypochlorite, sodium hypophosphite, disodium hydrogen phosphate, tocopherol, tocopherol acetate, arginine, butyl hydroxytoluene/Na-EDTA, acetylcysteine (N-acetylcysteine), butyl hydroxytoluene, allantoin, butyl hydroxyanisole, sodium carbonate, cysteamine and N-acetylcysteine.

Preferred antioxidants are compounds of the phenol type. Preferred phenolic compounds are, for example, butyl hydroxytoluene or butyl hydroxyanisole, as they completely prevent both delay in dissolution and yellowing. Other suitable products are: catechol, gallic acid or esters thereof, tocopherol, caffeic acid, hydroquinone monomethyl ether (MEHQ), nordihydroguaiaretic acid and resveratrol.

Other preferred antioxidants are thiolic compounds, such as N-acetylcysteine, cysteamine and thioglycolic acid.

Basic amino acids such as arginine and lysine are also preferred.

Preferred antioxidants are also alkali metal carbonates or alkali metal bicarbonates, in particular the sodium salts, preferably sodium carbonate.

Combinations with EDTA, in particular Na-EDTA or with citric acid, are also preferred.

N-Acetylcysteine, arginine, lysine, butyl hydroxytoluene, butyl hydroxytoluene/Na EDTA, and sodium carbonate or combinations thereof, are especially preferred.

All the stated compounds or classes of compounds can also be used in combination.

The antioxidants may be used in amounts of 0.01-30, preferably 0.1-20, especially preferably 0.5-12 wt. %, relative to the total amount of solid matter in the wet-binder preparation.

### Component C

Furthermore, as component C, the wet binders according to the invention may comprise plasticizers, preferably lipophilic plasticizers. Especially suitable plasticizers are tributyl citrate, acetyltributyl citrate, triacetin, triethyl citrate, acetyl triethyl citrate, diethyl sebacate and dibutyl sebacate.

The plasticizers may be used in amounts of 1 to 30, preferably 2 to 25, more preferably 5 to 20 wt. %, relative to the total amount of the solids content of the wet-binder preparation.

### Components D

The wet binders used in accordance with the invention for pharmaceutical administration forms may further comprise, as components D, at least one physiologically acceptable acid. This acid results in partial to complete salt formation with the basic polymer. In the case of complete salt formation, a polymer solution is formed.

Physiologically acceptable acids are the acids which it is known can be used in the sectors of pharmacy, food technology, and adjacent areas, particularly those listed in relevant pharmacopeias (e.g., Ph. Eur., USP, JP), food approval lists, and the books Fiedler, H. P., Lexikon der Hilfsstoffe für Pharmazie, Kosmetik and angrenzende Gebiete, 4th edn., Aulendorf: ECV-Editio-Cantor-Verlag, 1996, and P. H. Stahl and C. G. Stahl, Handbook of Pharmaceutical Salts, Helvetica Chimica Acta, 2002.

Examples of suitable acids are as follows: hydrochloric acid, sulfuric acid, phosphoric acid, carbonic acid, methanesulfonic acid, ethanesulfonic acid, acetic acid, maleic acid, fumaric acid, malonic acid, malic acid, succinic acid, citric acid, tartaric acid, lactic acid, benzoic acid, adipic acid, glycolic acid, propionic acid, salicylic acid, mandelic acid, glutamic acid and aspartic acid.

The acids can be used in amounts of 1 to 50 wt. %, preferably 2 to 35 wt. %, more particularly 5 to 25 wt. %, relative to the total weight of the solids content of the wet-binder preparation.

The amounts used are dependent in each case, according to the molecular weight of the acid used, on factors including the degree of neutralization that is to be achieved for component A). Where a component D) is used, then at least a degree of partial neutralization of 10 mol % of the basic groups of component A) is to be achieved, preferably more than 50 mol %, more preferably 90 to 100 mol %.

In one embodiment of the invention, wet-binder preparations used are aqueous dispersions which comprise only component A (method I).

According to a further embodiment, wet-binder preparations used are aqueous dispersions which in addition to component A) further comprise a component B) (method II).

According to a further, preferred, embodiment, the wet-binder preparations comprise a combination of components A) and C) (method III).

According to a further, preferred, embodiment, the aqueous dispersions comprise a combination of components A) and D), the aqueous dispersion undergoing transition, depending on the degree of neutralization, into an aqueous solution (method IV).

A further embodiment relates to a combination of components A), C) and D) (method V).

A further embodiment relates to a combination of components A), B) and C) (method VI).

A further embodiment relates to a combination of components A), B) and D) (method VII).

Furthermore, the invention also relates to an embodiment in which the wet binders comprise a combination of components A), B), C) and D) (method VIII).

The numerical figures in the table below refer to weight percentages.

| | | Component B, | Component C, | Component D, |
|---|---|---|---|---|
| | Compo- | if present: | if present: | if present: |
| | nent | methods II, VI, | methods III, V, | methods IV, V, |
| | A | VII, VIII | VIII | VII, VIII |
| | 1-45 | 0.01-30 | 1-30 | 1-50 |
| Preferred | 2-40 | 0.1-20 | 2-25 | 2-35 |
| Particularly | 5-30 | 0.5-20 | 5-20 | 5-25 |
| preferred | | | | |

The quantitative figures for component A relate to the total weight of the wet binder. The quantitative figures for components B), C) and D) relate to the total solids content of the wet binder.

The quantitative ranges, irrespective of whether they are not preferred, are preferred or are particularly preferred, can be combined freely in the different embodiments, depending on the profile of properties to be set for the granules and for the tablets obtainable from them. Thus, for example, if desired or necessary, the release stability can be adjusted via the variation of components B. By varying the amounts of plasticizer (component C)) and/or component D) (neutralizing acid) it is possible to adjust the mechanical properties and/or the release profile.

According to one preferred embodiment, the preparations comprise 2 to 35 wt. % of component D), relative to component A), and the fraction of component A), relative to the total weight of the dispersion, is 2 to 40 wt. %.

According to another preferred embodiment, the preparations comprise 2 to 25 wt. % of component C), relative to component A), and the fraction of component A), relative to the total weight of the dispersion, is 2 to 40 wt. %.

According to another preferred embodiment, the preparations comprise 0.1 to 20 wt. % of component B), relative to component A), and the fraction of component A), relative to the total weight of the dispersion, is 2 to 40 wt. %.

According to another preferred embodiment, the preparations comprise 2 to 25 wt. % of component C) and 2-35 wt. % of component D), relative to component A), and the fraction of component A), relative to the total weight of the dispersion, is 2 to 40 wt. %.

According to one particularly preferred embodiment, the preparations comprise 2 to 55 wt. % of component D), relative to component A), the fraction of component A), based on the total weight of the dispersion, is 5 to 30 wt. %.

According to another preferred embodiment, the preparations comprise 5 to 20 wt. % of component C), relative to component A), the fraction of component A), based on the total weight of the dispersion, is 5 to 30 wt. %.

According to another preferred embodiment, the preparations comprise 0.5 to 20 wt. % of component B), relative to component A), the fraction of component A), based on the total weight of the dispersion, is 5 to 30 wt. %.

According to another preferred embodiment, the preparations comprise 5 to 20 wt. % of component C) and 5-25 wt. % of component D), relative to component A), the fraction of component A), based on the total weight of the dispersion, is 5 to 30 wt. %.

In the case of the compositions identified above, the amounts of components A, B, C, D and dispersion medium and/or solvent add up to 100 wt. %. Dispersion media are water or mixtures of water and a water-miscible organic solvent.

The wet-binder preparations are produced, according to one embodiment of the invention, starting from an aqueous dispersion of component A. The incorporation of components B-D into the aqueous polymer dispersion can take place in general by stirred incorporation or intimate mixing. Both with solid and liquid components B-D, they can also be dissolved in water or diluted with water prior to their addition to the polymer dispersion.

In one particular embodiment, the polymers described (components A) are used as a solution in a purely organic solvent. For the preparation, the solid polymers are dissolved in a suitable solvent such as, for example, ethanol, isopropanol, methanol, acetone, methylene chloride, ethyl acetate, tetrahydrofuran, dioxane.

The preparation of an organic-aqueous solution may also be accomplished by adding a water-miscible solvent such as, for example, ethanol, isopropanol, methanol, acetone, tetrahydrofuran or dioxane to a polymer dispersion. Complete dissolution generally necessitates a solvent to polymer dispersion ratio of more than 1:1 to 25:1.

This organic or organic-aqueous solution can be used like a purely aqueous preparation for wet granulation.

The wet-binder preparations preferably do not receive any additional organic solvents.

In accordance with certain embodiments of the invention, the binders serve to produce pharmaceutical administration forms, especially pharmaceutical granules and tablets obtained by tabletting such granules. It is possible, surprisingly, to achieve a considerable increase in the activity of the wet binder if the basic polymers are neutralized or partly neutralized and/or admixed with plasticizers. The wet binders produce stable granules which are insensitive to abrasion and include a small fine fraction, and therefore do not dust very much. The tablets produced from these granules are very hard, have low sensitivity to abrasion, and disintegrate very rapidly. Consequently, the polymers of the invention ideally meet the requirements of a high-activity wet binder.

The wet binder can be prepared, for example, by stirred incorporation and intimate mixing of a polymer dispersion of the invention with at least one excipient.

The wet binder of the invention may be employed in aqueous dispersion or in fully neutralized form, as a solution, by means of pouring, dropwise addition or sprayed introduction. The moistened material is dried after the binder preparation has been added. In the case of fluidized-bed granulations, sprayed introduction and drying take place in parallel.

The wet binders according to the invention are suitable for dosage forms basically of any pharmaceutical active compounds, which can preferably be administered in isolated or protected form, such as antidepressants, beta blockers, antidiabetic agents, analgesics, antiphlogistics, antirheumatics, antihypotensives, antihypertensives, psychoactive drugs, tranquilizers, antiemetics, muscle relaxants, glucocorticoids, agents for the treatment of ulcerative colitis or Crohn's disease, antiallergic agents, antibiotics, antiepileptics, anticoagulants, antimycotics, antitussives, arteriosclerotic agents, diuretics, enzymes, enzyme inhibitors, gout agents, hormones and inhibitors thereof, cardiac glycosides, immunotherapeutic agents and cytokines, laxatives, antilipemic agents, gastrointestinal therapeutic agents, antimigraine agents, preparations of minerals, otologic agents, antiparkinsonian agents, thyroid therapeutic agents, spasmolytics, antiplatelet agents, vitamins, cytostatics and metastasis inhibitors, phytopharmaceuticals, chemotherapy agents, nutraceuticals, vitamins, carotinoids and amino acids.

Examples of suitable active compounds are: acarbose, nonsteroidal antirheumatics, cardiac glycosides, acetylsalicylic acid, virustatic agents, aclarubicin, aciclovir, cisplatin, actinomycin, α- and β-sympathomimetics, allopurinol, alosetron, alprostadil, prostaglandins, amantadine, ambroxol, amlodipine, methotrexate, 5-aminosalicylic acid, amitriptyline, amoxicillin, anastrozole, atenolol, atorvastatin, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cephalosporins, celetoxib, cetirizine and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglycic acid, coumarin and coumarin derivatives, cysteine, cytarabine, cyclophosphamide, cyclosporin, cyproterone, cytarabine, dapiprazole, desogestrel, desonide, dihydralazine, diltiazem, ergot alkaloids, dimenhydrinate, dimethylsulfoxide, dimeticone, dipyridamole, domperidone and domperidone derivatives, donepzil, dopamine, doxazosin, doxorubicin, doxylamine, dapiprazole, benzodiazepine, diclofenac, glycoside antibiotics, desipramine, econazole, ACE inhibitors, enalapril, ephedrine, epinephrin, epoetin and epoetin derivatives, morphinanes, calcium antagonists, irinotecan, modafinil, orlistat, peptide antibiotics, phenyloin, riluzole, risedronate, sildenafil, topiramate, macrolide antibiotics, esomeprazole, estrogen and estrogen derivatives, gestagen and gestagen derivatives, testosterone and testosterone derivatives, androgen and androgen derivatives, ethenzamide, etofenamate, etofibrate, fenofibrate, etofylline, etoposide, famciclovir, famotidine, felodipine, fentanyl, fenticonazole, gyrase inhibitors, fluconazole, fludarabine, flunarizine, fluorouracil, fluoxetine, flurbiprofen, ibuprofen, flutamide, fluvastatin, follitropin, formoterol, fosfomycin, furosemide, fusidic acid, galantamine, gallopamil, ganciclovir, gemfibrozil, gentamicin, ginkgo, St. John's-wort, glibenclamide, urea derivatives as oral antidiabetics, glucagon, glucosamine and glucosamine derivatives, glutathione, glycerol and glycerol derivatives, hypothalamus hormones, goserelin, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, iphosphamide, imipramine, indometacin, indoramin, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixene, metoclopramide, metoprolol, metronidazole, mianserin, miconazole, minocycline, minoxidil, misoprostol, mitomycin, mizolastine, moexipril, morphine and morphine derivatives; evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novamine sulfone, noscapine, nystatin, ofloxacin, olanzapine, olsalazine, omeprazole, omoconazole, ondansetron, orlistat, oseltamivir, oxaceprol, oxacillin, oxiconazole, oxymetazoline, pantoprazole, paracetamol, paroxetine, penciclovir, oral penicillin, pentazocine, pentifylline, pentoxifylline, perphenazine, pethidine, plant extracts, phenazone, pheniramine, barbituric acid derivatives, phenylbutazone, phenyloin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirole, rosiglitazone, roxatidine, roxithromycin, ruscogenin, rutoside and rutoside derivatives, sabadilla, salbutamol, salmeterol, scopolamine, selegiline, sertaconazole, sertindole, sertraline, silicates, simvastatin, sitosterol, sotalol, spaglumic acid, sparfloxacin, spectinomycin, spiramycin, spirapril, spironolactone, stavudine, streptomycin, sucralfate, sufentanil, sulbactam, sulfonamides, sulfasalazine, sulpiride, sultamicillin, sultiame, sumatriptan, suxamethonium chloride, tacrine, tacrolimus, talinolol, tamoxifen, taurolidine, tazarotene, tegaserod, temazepam, teniposide, tenoxicam, terazosin, terbinafine, terbutaline, terfenadine, terlipressin, tertatolol, tetracyclines, tetryzoline, theobromine, theophylline, butizine, thiamazole, phenothiazines, thiotepa, tiagabine, tiapride, propionic acid derivatives, ticlopidine, timolol, tinidazole, tioconazole, thioguanine, thioxolone, tiropramide, tizanidine, tolazoline, tolbutamide, tolcapone, tolnaftate, tolperisone, topotecan, torasemide, antiestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpine, troxerutin, tulobuterol, tyramine, tyrothricin, urapidil, ursodeoxycholic acid, chenodeoxycholic acid, valaciclovir, valdecoxib, valproic acid, vancomycin, vecuronium chloride, venlafaxine, verapamil, vidarabine, vigabatrin, viloxazine, vinblastine, vincamine, vincristine, vindesine, vinorelbine, vinpocetine, viquidil, warfarin, xantinol nicotinate, xipamide, zafirlukast, zalcitabine, zanamivir, zidovudine, zolmitriptan, zolpidem, zopiclone, zotepine and the like.

The active compounds can, if desired, also be used in the form of their pharmaceutically acceptable salts or derivatives, and in the case of chiral active compounds, both optically active isomers and racemates or diastereoisomeric mixtures can be used. If desired, the compositions according to the invention can also comprise two or more pharmaceutical active compounds.

In accordance with the invention the granules can be packed in capsules, sachets or bulk-product packs such as powder bottles made of glass or plastic, for example. The granules are frequently then compressed to form tablets. Depending on type, the tablets may release the active compound quickly or with a delay, and they may also be processed further to form film-coated tablets. As a result of the insolubility of the polymers in the saliva, the bitterness and poor taste of pharmaceuticals is often hidden. This means that, through granulation, a taste-masking effect can be achieved. Nevertheless, within the stomach, there is very rapid release of the active compound from the administration form. The use of the polymers of the invention in granulation therefore has a number of advantages: stable granules and stable tablets, high fluidity, good taste masking, rapid release of active compound in the stomach.

With the polymers of the invention it is possible, surprisingly, to process even pharmaceuticals that are of low solubility in water to give quick-release administration forms. Pharmaceuticals that are of low solubility in water are those pharmaceuticals for which at least 100 parts of water are needed in order to dissolve one part of pharmaceutical. This also encompasses virtually insoluble pharmaceuticals, for which at least 10,000 parts of water are needed in order to dissolve one part of pharmaceutical.

For producing tablets or other administration or dosage forms, the granules can also be mixed with further, customary, pharmaceutical excipients. These are substances from the classes of the fillers, plasticizers, solubility enhancers, binders, silicates, as well as disintegrants, adsorbents, lubricants, flow agents, colorants, stabilizers such as antioxidants, wetting agents, preservatives, mold release agents, flavors or sweeteners, preferably fillers, disintegrants and lubricants.

Fillers which can be added include, for example, inorganic fillers such as oxides of magnesium, aluminum, silicon, titanium carbonate or calcium carbonate, calcium or magnesium phosphates, or organic fillers such as lactose, sucrose, sorbitol or mannitol.

Examples of suitable plasticizers include triacetin, triethyl citrate, glycerol monostearate, low molecular weight polyethylene glycols or poloxamers.

Suitable additional solubilizers include surface-active substances having an HLB (hydrophilic lipophilic balance) value of more than 11, examples being hydrogenated castor oil ethoxylated with 40 ethylene oxide units (Cremophor® RH 40), castor oil ethoxylated with 35 ethylene oxide units (Cremophor® eL), polysorbate 80, poloxamers or sodium lauryl sulfate.

Lubricants which can be used include stearates of aluminum, calcium, magnesium and tin, and also magnesium silicate, free fatty acids such as stearic acid, for example, poloxamers, sodium stearyl fumarate, and the like.

Examples of flow agents which can be used are talc or colloidal silicon dioxide.

An example of a suitable binder is microcrystalline cellulose.

Disintegrants may be crosslinked polyvinylpyrrolidone, crosslinked sodium carboxymethylcellulose, L-HPC or crosslinked sodium carboxymethylstarch.

Possible stabilizers are ascorbic acid or tocopherol.

Colorants are, for example, iron oxides, titanium dioxide, triphenylmethane dyes, azo dyes, quinoline dyes, indigo tin dyes, carotenoids, in order to color the administration forms, opacifiers such as titanium dioxide or talc, in order to reduce the transparency, and in order to save on colorants.

The examples which follow are intended to illustrate certain embodiments of the invention in more detail, without restricting it.

### EXAMPLES

### Abbreviations Used

d: days
s: seconds
DE: fully demineralized
RRSB: Rosin, Rammler, Sperling and Bennett particle size distribution; the average particle size corresponds to the particle size with a sieve residue of 36.8% (Ingfried Zimmermann, Pharmazeutische Technologie, Springer Verlag, 1998, 276-278). Friability and fracture strength were determined in accordance with European Pharmacopoeia 7.0 Volume 1.
Granulac® 230: lactose, fine powder
Kollidon® CL: crosslinked polyvinylpyrrolidone (Crospovidon), average particle size 120 μm
Kollidon® 30: polyvinylpyrrolidone with K value of 30 (at 1% strength by weight in water)

All percentages relate to wt. % unless stated otherwise.

### Cationic Polymers:

The production of the polymers is carried out as in example 1 of WO 2009/016258.

Polymer A: methyl methacrylate/diethylaminoethyl methacrylate, weight ratio 60:40,
Polymer B: methyl methacrylate/diethylaminoethyl methacrylate, weight ratio 55:45
Polymer C: methyl methacrylate/diethylaminoethyl methacrylate, weight ratio 53:47

The K values measured 1 by weight in N-methylpyrrolidone were:

polymer A: 50.1
polymer B: 48.1
polymer C, 52.4

The polymers were used as 30 wt. % aqueous dispersions with a pH of 9+/−0.3. The average particle size of the primary dispersion was 110 nm.

The release from administration forms was determined using the device with paddle stirrer described under “Dissolution” in the US Pharmacopeia (USP 32).

### Example 1

### Granulation of an Excipients Mixture in a Vertical Mixer

### Production of the Binder Preparations

### 1.1. Kollidon® 30 (Comparative Example)

45 g of Kollidon® 30 were dissolved in 255 g of water.

### 1.2. Cationic Polymer A

150 g of the aqueous dispersion of the cationic polymer A were admixed with 150 g of water.

### 1.3. Fully Neutralized Cationic Polymer A

150 g of the aqueous dispersion were diluted with 56.5 g of water and then admixed with 97 ml of a 1N HCl solution. The 97 ml of the 1N HCl solution comprised 3.5 g of HCl.

1.4. Cationic Polymer A with 15% Plasticizer, Relative to the Polymer

6.75 g of plasticizer were diluted with 150 g of water and then added slowly to 150 g of the 30% dispersion.

| Formula: | Initial mass | |
|---|---|---|
| Calcium hydrogen phosphate | 525 | g |
| Granulac 230 | 75 | g |
| Binder preparation | | |
| Binder | 12.75 | g |
| Water | 72.25 | g |

Calcium hydrogen phosphate and Granulac 230 were weighed out into a Diosna stirring vessel. This mixture was mixed for 1 minute in a Diosna mixer (400 rpm stirrer/2200 rpm chopper). 85 g of the 15.0% binder solution were added by means of a syringe over the course of 45 s, with continual stirring, to the stirring container (400 rpm stirrer/2200 rpm chopper). Following the addition of the binder solution to the mixer, there was a mixing time of 3 min. Thereafter the moistened material was passed through a sieve with a mesh size of 0.8 mm, and the wet granules were dried on racks in air at 25° C. for at least 12 hours. After that, the dried granules were again passed through a sieve with a mesh size of 0.8 mm.

A tabletting mixture was subsequently produced from the granules:

| Formula: tabletting mixture | Proportion |
|---|---|
| Granules | 96.5% |
| Kollidon ® CL | 3% |
| Magnesium stearate | 0.5% |

The disintegrant and lubricant were passed through a sieve with a mesh size of 0.8 mm and then added to the granules. The entire quantity of material was introduced into a glass bottle, which was sealed, and mixed for 5 minutes in a Turbula mixer. The tabletting mixture was tableted on an eccentric press (from Korsch, XP 1) at 10 kN to form biplanar tablets having a diameter of 10 mm. The tablet weight was 500 mg.

### Granules and Tablet Properties

| | | | | 1.4. |
|---|---|---|---|---|
| | | 1.2. | | cationic |
| | | cationic | 1.3. cationic | polymer |
| | 1.1. Kollidon ® | polymer | polymer A | A with |
| | 30/comparative | A | neutralized | triacetin |
| Average | 300 | 266 | 341 | 280 |
| particle size x′ | | | | |
| by RRSB | | | | |
| (μm) | | | | |
| Fine fraction | 12.5 | 20.8 | 8.6 | 14.1 |
| <125 μm (%) | | | | |
| Friability of | 31.6 | 58.2 | 26.0 | 30.3 |
| granules after | | | | |
| 3 min (%) | | | | |
| Breaking | 19 | 24 | 34 | 27 |
| strength of | | | | |
| tablets under | | | | |
| pressing force | | | | |
| of 10 kN (N) | | | | |
| Friability of | 2.5 | 2.2 | 0.48 | 1.15 |
| tablets (%) | | | | |
| Disintegration | 23 | 10 | 22 | 10 |
| time in | | | | |
| phosphate | | | | |
| buffer pH 6.8 | | | | |
| (s) | | | | |

The friability of the granules was determined using an LPS 200 MC air-jet sieve from Rhewum. The fine fraction <125 μm was first removed by sieving, at a low air throughput of 20 m³/h and a sieving time of 1 min. Subsequently, the residue was weighed and treated with an airflow of 75 m³/h over 3 min (severe load). The residue which remained in this case was weighed again. The friability is given by the difference, and is expressed in percent of the residue following sieve removal of the fine fraction.

### Example 2

### Granulation of an Active Compound/Excipient Mixture in a Vertical Mixer

The production of the wet-binder preparation based on polymer B took place in the same way as in example 1.

| Formula: granulation | Initial mass | |
|---|---|---|
| Calcium hydrogen phosphate | 787.5 | g |
| Vitamin C | 112.5 | g |
| Binder solution | | |
| Binder | 13.5 | g |
| Water | 76.5 | g |

Calcium hydrogen phosphate and vitamin C were weighed out into a Diosna stirring vessel. This mixture was mixed for 1 minute in a Diosna mixer (400 rpm stirrer/2200 rpm chopper). 90 g of the 15.0% binder solution were added by means of a syringe over the course of 45 s, with continual stirring, to the stirring container (400 rpm stirrer/2200 rpm chopper). Following the addition of the binder solution to the mixer, there was a mixing time of 3 min. Where necessary, water was added in order to produce optimum moistening throughout the mixture. Thereafter the moistened material was passed through a sieve with a mesh size of 0.8 mm, and the wet granules were dried on racks in air at 25° C. for at least 12 hours. After that, the dried granules were again passed through a sieve with a mesh size of 0.8 mm.

A tabletting mixture was subsequently produced from the granules:

| Formula: tabletting mixture | Proportion |
|---|---|
| Granules | 96.5% |
| Kollidon ® CL | 3% |
| Magnesium stearate | 0.5% |

The disintegrant and lubricant were passed through a sieve with a mesh size of 0.8 mm and then added to the granules. The entire quantity of material was introduced into a glass bottle, which was sealed, and mixed for 5 minutes in a Turbula mixer. The tabletting mixture was tableted on an eccentric press (from Korsch, XP 1) at 18 kN to form biplanar tablets having a diameter of 10 mm. The tablet weight was 500 mg.

| | | | | 2.4. |
|---|---|---|---|---|
| | 2.1. | 2.2. | 2.3. | cationic |
| | Kollidon ® | cationic | cationic | polymer B |
| | 30/com- | polymer | polymer B | with |
| | parative | B | neutralized | triacetin |
| Average particle size x′ | 285 | 232 | 328 | 282 |
| by RRSB (μm) | | | | |
| Fine fraction <125 μm | 5.9 | 22.3 | 5.5 | 6.9 |
| (%) | | | | |
| Friability of granules | 25.4 | 26.5 | 20.1 | 22.8 |
| after 3 min (%) | | | | |
| Breaking strength of | 38 | 49 | 52 | 47 |
| tablets under pressing | | | | |
| force of 18 kN (N) | | | | |
| Friability of tablets (%) | 0.45 | 0.65 | 0.39 | 0.67 |
| Disintegration time in | 48 | 26 | 51 | 30 |
| 0.1 N HCl (s) | | | | |
| Release of active | >90 | >90 | >90 | >90 |
| compound after 60 min | | | | |
| in 800 ml of 0.1 N HCl | | | | |
| at 50 revolutions/min | | | | |
| (%) | | | | |

### Example 3

### Granulation of Vitamin C in the Fluidized Bed

### Preparation of the Binder Solution for Granulation in the Fluidized Bed

### 3.1. Kollidon® 30 (for Comparison)

75 g of Kollidon® 30 were dissolved in 675 g of water.

### 3.2. Cationic Polymer C

250 g of the 30% dispersion of the cationic polymer were admixed with 500 g of water.

### 3.3. Fully Neutralized Cationic Polymer C

250 g of the 30% dispersion were diluted with 344 g of water and then admixed with 162 ml of a 1N HCl solution with stirring. The 162 ml of the 1N HCl solution comprise 5.9 g of HCl.

3.4. Cationic Polymer C with 15% Plasticizer, Relative to the Polymer

11.25 g of plasticizer were diluted with 500 g of water and then added slowly to 250 g of the 30% dispersion.

### Production Parameters of the Granulation of Vitamin C in the Fluidized Bed

| Production parameters | | |
|---|---|---|
| Top Spray | | |
| Nozzle | 1 | mm |
| Feed air temperature | 55° C. | |
| Product temperature | 30° C. | |
| Volume flow rate | 87 | m³/h |
| Spraying pressure | 1.5 | bar |
| Spraying rate | 20 | g/min |
| Formula/Granulation | Initial mass | |
| Vitamin C | 1940 | g |
| Binder solution | | |
| Binder | 60 | g |
| Water | 540 | g |
| Amount applied | 600 | g |

Vitamin C (in the form of a powder with a particle size of 45 μm) was weighed out into the granulation vessel of the Glatt WSG GPC G3 and heated for 5 minutes. The volume flow rate was 87 m³/h and the temperature of the feed air was 55° C. 600 g of the 10.0% strength binder solution were sprayed onto the vitamin C over the course of 30 min. After spraying had taken place, the granules were dried at a feed air temperature of 55° C. for 5 min. The dry granules were then again passed through a sieve with a mesh size of 0.8 mm.

A tabletting mixture was subsequently produced from the granules:

| Formula: tabletting mixture | Composition = 100% |
|---|---|
| Granules | 96% |
| Kollidon ® CL | 3% |
| Magnesium stearate | 1% |

The disintegrant and lubricant were passed through a sieve with a mesh size of 0.8 mm and then added to the granules. The entire material was introduced into a glass bottle, which was sealed, and mixed for 5 minutes in a Turbula mixer.

The tabletting mixture was tableted on an eccentric press (from Korsch, XP 1) at 18 kN to form biplanar tablets having a diameter of 10 mm. The tablet weight was 300 mg.

| | | | | 3.4. |
|---|---|---|---|---|
| | 3.1. | 3.2. | 3.3. | cationic |
| | Kollidon ® | cationic | cationic | polymer C |
| | 30/com- | polymer | polymer C | with |
| | parative | C | neutralized | triacetin |
| Average particle size | 93 | 93 | 127 | 95 |
| d(4.3) by laser | | | | |
| diffraction (μm) | | | | |
| Fine fraction <125 | 53.0 | 57.6 | 38.3 | 42.7 |
| μm by sieving (%) | | | | |
| Friability of granules | 76.4 | 60.3 | 64.9 | 65.9 |
| after 3 min (%) | | | | |
| Breaking strength of | 87 | 96 | 98 | 100 |
| tablets under | | | | |
| pressing force of 18 | | | | |
| kN (N) | | | | |
| Friability of tablets | 0.30 | 0.24 | 0.10 | 0.15 |
| (%) | | | | |
| Release of active | >90 | >90 | >90 | >90 |
| compound after 20 | | | | |
| min in 800 ml of 0.1 | | | | |
| N—HCl at 50 | | | | |
| revolutions/min (%) | | | | |

### Example 4

### Granulation of Paracetamol in Fluidized Bed by Means of Organic-Aqueous Polymer Solution

### Preparation of the Binder Solution for Granulation in the Fluidized Bed

500 g of a 30% aqueous dispersion of the cationic polymer C were admixed slowly with 1000 g of isopropanol, with stirring, and stirring was continued until a clear solution had formed.

### Production Parameters: Granulation of Paracetamol in the Fluidized Bed

| Production parameters | | |
|---|---|---|
| Top Spray | | |
| Nozzle | 1 | mm |
| Feed air temperature | 40° C. | |
| Product temperature | 33° C. | |
| Volume flow rate | 87 | m³/h |
| Spraying pressure | 1.5 | bar |
| Spraying rate | 20 | g/min |
| Formula/Granulation | Initial mass | |
| Paracetamol | 1880 | g |
| Binder solution | | |
| Binder | 120 | g |
| Water/isopropanol | 1080 | g |
| Amount applied | 1200 | g |

Paracetamol was weighed out into the granulation vessel of the Glatt WSG GPC G3 and heated for 5 minutes. The volume flow rate was 87 m³/h and the temperature of the feed air was 40° C. 1200 g of the 10.0% strength binder solution were sprayed onto the paracetamol over the course of 30 min. After spraying had taken place, the granules were dried at a feed air temperature of 40° C. for 5 min. The dry granules were then again passed through a sieve with a mesh size of 0.8 mm.

A tabletting mixture was subsequently produced from the granules:

| Formula: tabletting mixer | Quantitative proportion |
|---|---|
| Granules | 85% |
| Kollidon ® CL | 3% |
| Microcrystalline cellulose | 10% |
| Polyethylene glycol, fine powder | 2% |

Kollidon® CL, microcrystalline cellulose and polyethylene glycol were passed through a sieve with a mesh size of 0.8 mm and then added to the granules. The entire material was introduced into a glass bottle, which was sealed, and mixed for 5 minutes in a Turbula mixer.

The tabletting mixture was tableted on an eccentric press (from Korsch, XP 1) at 25 kN to form biplanar tablets having a diameter of 12 mm. The tablet weight was 625 mg.

Tablet properties

| | |
|---|---|
| Average particle size | 161 |
| d(4.3) by laser | |
| diffraction (μm) | |
| Breaking strength of | 148 |
| tablets at 25 kN | |
| pressing force (N) | |
| Friability of tablets (%) | 0.14 |
| Release of active | >90 |
| compound after 45 min | |
| in 800 ml of 0.1 N—HCl | |
| at 50 revolutions/min | |
| (%) | |

The tablets did not have a bitter taste.

What is claimed is:

1. A method for preparing active compound-containing granules comprising using a matrix binder comprising a copolymer having basic amino groups obtained by radical polymerization of:
a) N,N-diethylaminoethyl methacrylate; and
b) at least one radically polymerizable compound, selected from esters of α,β-ethylenically unsaturated mono- and dicarboxylic acids with C₁-C₈ alkanols.

2. The method according to claim 1, wherein the matrix binder comprises:
a) 43 to 47 wt. % N,N-diethylaminoethyl methacrylate, relative to the total weight of the monomers used for polymerization; and
b) 53 to 57 wt. % methyl methacrylate, relative to the total weight of the monomers used for polymerization.

3. The method according to claim 1, wherein the matrix binder is in the form of a wet-binder preparation.

4. The method according to claim 2, wherein the matrix binder is in the form of an aqueous or aqueous-organic dispersion or solution.

5. The method according to claim 1, wherein the matrix binder further comprises one or more of:
ii) one or more antioxidants as component B;
iii) one or more plasticizers as component C; and
iv) one or more physiologically acceptable acids as component D.

6. The method according to claim 5, wherein the one or more antioxidants comprise one or more of N-acetylcysteine, arginine, lysine, butyl hydroxytoluene, butyl hydroxytoluene/Na EDTA, and sodium carbonate.

7. The method according to claim 5, wherein the one or more plasticizers comprise one or more of tributyl citrate, acetyl tributyl citrate, triacetin, triethyl citrate, acetyl triethyl citrate, diethyl sebacate, and dibutyl sebacate.

8. The method according to claim 5, wherein the one or more physiologically acceptable acids comprise one or more of hydrochloric acid, sulfuric acid, phosphoric acid, carbonic acid, methanesulfonic acid, ethanesulfonic acid, acetic acid, maleic acid, fumaric acid, malonic acid, malic acid, succinic acid, citric acid, tartaric acid, lactic acid, benzoic acid, adipic acid, glycolic acid, propionic acid, salicylic acid, mandelic acid, glutamic acid, and aspartic acid.

9. The method according to claim 1, wherein the basic amino groups are present in partly or wholly neutralized form.

10. A matrix binder comprising, as component A, a copolymer having basic amino groups obtained by radical polymerization of:
a) N,N-diethylaminoethyl methacrylate; and
b) at least one radically polymerizable compound, selected from esters of α,β-ethylenically unsaturated mono- and dicarboxylic acids with C₁-C₈ alkanols.

11. The matrix binder according to claim 10, wherein the matrix binder comprises:
a) 43 to 47 wt. % N,N-diethylaminoethyl methacrylate, relative to the total weight of the monomers used for polymerization; and
b) 53 to 57 wt. % methyl methacrylate, relative to the total weight of the monomers used for polymerization.

12. The matrix binder according to claim 10, wherein the matrix binder is in the form of an aqueous or organic-aqueous dispersion or solution.

13. The matrix binder according to claim 10, wherein the matrix binder comprises:
i) 1 to 45 wt. % of component A;
ii) 0 to 10 wt. % of one or more antioxidants as component B;
iii) 0 to 15 wt. % of one or more plasticizers as component C;
iv) 0 to 35 wt. % of one or more physiologically acceptable acids as component D.

14. The matrix binder according to claim 13, wherein the matrix binder comprises:
i) 5 to 30 wt. % of component A;
ii) 0 to 5 wt. % of component B;
iii) 0 to 8 wt. % of component C;
iv) 0.1 to 20 wt. % of components D.

15. The matrix binder according to claim 13, wherein the matrix binder comprises:
i) 5 to 30 wt. % of component A;
ii) 0 to 5 wt. % of component B;
iii) 0.1 to 8 wt. % of component C;
iv) 0 to 20 wt. % of components D.

16. The matrix binder according to claim 10, wherein the basic amino groups of component A are present in partly or wholly neutralized form.

17. An active compound-containing administration form comprising at least one active compound-containing granule obtained by the method according to claim 1.

18. An active compound-containing administration form comprising at least one active compound-containing granule obtained by the method according to claim 2.

19. An active compound-containing administration form comprising at least one active compound-containing granule obtained by the method according to claim 5.

20. An active compound-containing administration form comprising at least one active compound-containing granule obtained by the method according to claim 9.
